(19) United States
(12) Patent Application Publication
(10) Pub. No.: US 2018/0282700 A1
(43) Pub. Date: Oct. 4, 2018

(54) CELL CULTURE MODEL OF VASCULAR CALCIFICATION

(71) Applicant: Medizinische Universität Wien, Wien (AT)

(72) Inventors: Karin Macfelda, Wien (AT); Barbara Kapeller, Wien (AT); Alexander Holly, Wien (AT); Roman Lieber, Wien (AT)

(73) Assignee: Medizinische Universität Wien, Wien (AT)

(21) Appl. No.: 15/768,542

(22) PCT Filed: Oct. 13, 2016
(86) PCT No.: PCT/EP2016/074555

§ 371 Date: Apr. 14, 2018

(30) Foreign Application Priority Data
Oct. 15, 2015 (EP) 15189971.3

(51) Int. Cl. C12N 5/071; G01N 33/50

(52) U.S. Cl. CPC C12N 5/0691; G01N 33/5064; G01N 33/5061

(57) ABSTRACT

The present invention relates to a cell culture model of vascular calcification, comprising a first type of cultured vascular smooth muscle cells and a second type of cultured vascular smooth muscle cells, wherein the first type and the second type of vascular smooth muscle cells originate from blood vessels having a different diameter, e.g., from the human aorta and from a human coronary artery, respectively. This model allows to generate and analyze vascular calcification processes, including early calcification processes, in an advantageously simple and cost-effective way and with high sensitivity. It can further be used to identify and examine compounds capable of halting, inhibiting or reversing such vascular calcification processes.

The present invention relates to a cell culture model of vascular calcification, comprising a first type of cultured vascular smooth muscle cells and a second type of cultured vascular smooth muscle cells, wherein the first type and the second type of vascular smooth muscle cells originate from blood vessels having a different diameter, e.g., from the human aorta and from a human coronary artery, respectively. This model allows to generate and analyze vascular calcification processes, including early calcification processes, in an advantageously simple and cost-effective way and with high sensitivity. It can further be used to identify and examine compounds capable of halting, inhibiting or reversing such vascular calcification processes.

Vascular calcification is currently recognized as a cell regulated process caused among other things by loss of calcification inhibitors and involving osteoblast/chondroblast-like changes in vascular cell gene expression patterns and matrix development.

Pathological vascular calcification often is the beginning of severe diseases, which affect the vasculature and consequently also organs such as heart and kidney amongst others. It is known that nearly half the deaths in dialysis patients are due to cardiovascular disease. Vascular calcification, as a result of metabolic diseases such as diabetes, can also lead to severe diseases and be correlated with mortality.

As it is known that chronic kidney diseases are associated with mineral and bone disorders (Schwarz U et al., Nephrology Dialysis Transplantation, 15:218-23, 2000; Jehle P M & Deuber H J, Renale Osteopathie, Thieme, Stuttgart, N.Y., pages 3-13, 2001; Block G A et al., J Am Soc Nephrol, 15:2208-18, 2004; London G M, Clin J Am Soc Nephrol, 4(2):254-7, 2009) the term “chronic kidney disease—mineral and bone disorder” (CKD-MBD) was established in 2006 (Moe S et al., Kidney Int, 69:1945-53, 2006). The diagnosis CKD-MBD includes bone metabolism disorders (Nawroth P et al., Med Klin, 98:437-46, 2003) and metabolic disorders (vitamin D, calcium, phosphate, parathormone, etc.) as well as extra-osseous and especially cardiovascular calcification processes (Ketteler M et al., Dtsch med Wochenschr, 133(37):1844-7, 2008; Anavekar N S et al., N Engl J Med, 351:1285-95, 2004).

Phosphate is one of the most potent activators of vascular calcification processes (Lhotta K. J Klin Endokrinol Stoffw, 4(4):20-3, 2011) which causes vessel damages (Shanahan C M et al., Circulation, 100(21):2168-76, 1999). Additionally it has been shown in vitro and in vivo that an increased phosphate uptake leads to a remarkable endothelial dysfunction within the vascular system (Shuto E, et al. JASN, 20:1504-12, 2009).

An increased extracellular phosphate concentration, calcium and calcium-phosphate-products cause a higher intracellular phosphate uptake (Shanahan C M et al., Circulation, 100(21):2168-76, 1999) which is regulated by Pit-1, a sodium-depended co-transporter (Lau W L et al., Thromb Haemost, 104(3):464-70, 2010). A high concentration of this phosphate-transporter is detected within the cell membrane of vascular smooth muscle cells after treatment with inorganic phosphate (Villa-Bellosta R et al., Pflugers Arch, 458(6):1151-61, 2009). This indicates that extracellular phosphate regulates the deposition in smooth muscle cells indirectly (Zickler D, Dissertation, Medizinische Fakultät Charité-Universitätsmedizin Berlin, 2010). Foscarnet, an inhibitor of the co-transporter Pit-1, can block the phosphate uptake and further prevent the osteogenic differentiation by inhibiting the extracellular phosphate and calcium precipitation (Schwarz C, Wiener klinisches Magazin, 4:S38-43, 2009).

Molecular studies show that calcification processes can also be modulated and increased via intracellular regulations (Demer L L et al., Circulation, 117(22):2938-48, 2008). Under certain pathophysiological conditions such as diabetes mellitus or chronic kidney disease, smooth vascular muscle cells, vascular precursor cells or calcifying vascular cells get osteogenic characteristics which are regulated by the Cbfa1 signaling pathway and other signal transduction pathways (FGF, TGFβ, Notch, Hedgehog or Wnt).

Vascular calcification processes lead, beyond the deposition of tricalcium phosphate crystals, to metaplastic changes of smooth vascular muscle cells. Thereby, different modifications at the transcriptional level induce a loss of typical smooth muscle markers as well as the expression of osteochondrogenic proteins (Steitz S A et al., Circ Res, 89(12):1147-54, 2001; Moe Set al., Kidney Int, 69:1945-53, 2006).

Against calcification processes, vessels normally produce mineralization inhibitors such as pyrophosphate (O'Neill W C, Circ Res, 99(2):e2, 2006). Furthermore, a wide range of endogenic, physiological existing inhibitors of calcification have already been identified: Matrix gla-protein (MGP), fetuin A, osteopontin and osteoprotegerin (OPG) play a decisive role in the inhibition of mineral deposition (Schoppet M et al., Kidney Int, 73(4):384-90, 2008).

Some of these proteins are already known as disease and mortality markers in the clinical field (Bieglmayer C et al., J Miner Stoffwechs, 13(3):82-7, 2006). With the help of osteoprotegerin (OPG), matrix gla-protein (MGP), fetuin A and alkaline phosphatase (AP) the degree of vascular calcification can be estimated. Moreover, these biomarkers are used for indirect measurement of coronary calcification due to the fact that multiplanar computer tomography, a gold standard method, is very expensive and not permanently available. The temporal expression of biomarkers during progress of disease has essential prognostic value as well. Known calcification biomarkers provide an indication of occurring calcification processes but still cannot make a statement about triggering factors.

Calcification and/or the examination of calcification processes have been described, e.g., in: Shioi A et al., Arterioscler Thromb Vasc Biol, 15:2003-9, 1995; Wada T et al., Circ Res, 84:166-78, 1999; Giachelli C M, J Am Soc Nephrol, 14(9 Suppl 4):5300-4, 2003; Jono S et al., Circ Res, 87:e10-e17, 2000; Hsu H H et al., Lipids Health Dis, 7:2, 2008; Alves R D et al., BMC Genomics, 15:965, 2014; Yang H et al., Kidney Int, 66(6):2293-9, 2004; Shanahan C M et al., Circulation, 100(21):2168-76, 1999; Hénaut L et al., Cardiovasc Res, 101(2):256-65, 2014; EP-A-2479261; U.S. Pat. No. 6,812,034; US 2003/0027211; WO 2008/060139; and WO 2008/060156.

Clinical studies have furthermore demonstrated that calcification processes can be antagonized by using synthetic inhibitors such as bisphosphonates (Cremers S et al., Bone, 49(1):42-9, 2011). However, these observations have not been implemented in in-vitro models to date.

The addition of exogenous ATP to calcified cells also leads to a decreased calcification level (Prodoscimo A D et al., Am J Physiol Cell Physiol, 298:C702-C713, 2010), and microcurrent application on cells is able to increase intracellular ATP production. The effect of electrical stimuli on tissues and pathological processes has been the subject of considerable experimental attention. Electrical stimulation in various forms has been shown to enhance wound healing processes and tumor growth retardation (Cheitlin M D et al., Circulation, 95:1686-744, 1997; Slama M et al., Am J Physiol Heart Circ Physiol, 286:H181-5, 2004). Exposure to electrical as well as electromagnetic fields has profound effects on cellular function such as proliferation and cell division (King N & O'Connell J, RT-PCR Protocols (Methods in Molecular Biology). First edition, Humana Press, New York, 2002).

As of recently, calcification processes can be diagnosed by using cost-intensive methods (e.g., multiplanar computer tomography, intravascular ultrasound), but it is still impossible to show the beginning of calcification processes. It would therefore be highly desirable to provide a model system with easily detectable parameters which give quick and cost-efficient information about the beginning and the progression of vascular calcification processes.

It is thus an object of the present invention to provide a model system that allows generating and examining calcification processes, particularly beginning (early) calcification processes, in an in vitro environment in a rapid and cost-effective way. In the context of the present invention, it was contemplated that beginning vascular calcification processes proceed differently depending on the vessel size. This finding is utilized in the vascular calcification model provided in accordance with the invention, as described further below, in which the behavior of vascular smooth muscle cells isolated from blood vessels of different size, such as, e.g., aorta and coronary artery, are compared. The beginning and progression of calcification can be detected in a particularly rapid and quantitative manner by detecting the formation of hydroxyapatite. The cell culture model of vascular calcification according to the present invention furthermore provides a highly effective approach for identifying novel calcification inhibitors as well as investigating the effects and mode of action of known calcification inhibitors, including bisphosphonates such as zoledronate, ibandronate or etidronate.

Accordingly, the present invention provides a cell culture model of vascular calcification, comprising a first type of cultured vascular smooth muscle cells and a second type of cultured vascular smooth muscle cells, wherein the first type and the second type of vascular smooth muscle cells originate from blood vessels having a different diameter. This cell culture model of vascular calcification can also be referred to as an in vitro cell culture model of vascular calcification.

The present invention likewise relates to the use of a first type of cultured vascular smooth muscle cells and a second type of cultured vascular smooth muscle cells as a cell culture model of vascular calcification (or as an in vitro cell culture model of vascular calcification), wherein the first type and the second type of vascular smooth muscle cells originate from blood vessels having a different diameter.

The cell culture model according to the invention is highly advantageous in that it is hydroxyapatite sensitive, inexpensive, quick and easy to use, and also in that it allows the detection of very early on-going calcification processes, the detection of new biomarkers which indicate the stage of calcification, the development of new therapeutics and diagnostics, and the uncovering of the mechanism of action of known as well as novel calcification inhibitors. The use of vascular smooth muscle cells from blood vessels of different size provides an advantageously high sensitivity in the detection of vascular calcification, particularly since calcification can typically be detected earlier in cells from blood vessels of smaller size, which allows the identification of biomarkers expressed very early in the vascular calcification process.

The first type and the second type of vascular smooth muscle cells to be used in accordance with the invention originate from blood vessels having a different diameter, i.e. have been isolated from blood vessels having a different diameter, but are otherwise not particularly limited. Preferably, they are human cells, particularly primary human vascular smooth muscle cells. The size of blood vessels in human beings varies enormously, and ranges from a diameter of about 25 mm in the aorta to about 8 μm in the capillaries. It is preferred that the first type of vascular smooth muscle cells originates from a blood vessel (particularly a human blood vessel) having a diameter that is at least about 3 times as large as the diameter of the blood vessel (particularly a human blood vessel) from which the second type of vascular smooth muscle cells originates. More preferably, the first type of vascular smooth muscle cells originates from a blood vessel having a diameter that is at least about 5 times (even more preferably at least about 10 times, even more preferably at least about 20 times, even more preferably at least about 30 times, even more preferably at least about 50 times, even more preferably at least about 100 times, even more preferably at least about 200 times, even more preferably at least about 300 times, and yet even more preferably at least about 400 times; or, e.g., at least about 500, 600, 700, 800, 900 or 1000 times) as large as the diameter of the blood vessel from which the second type of vascular smooth muscle cells originates. It is to be understood that the diameter of any particular blood vessel, as specified herein, refers to the inner diameter of the respective blood vessel, particularly at the site of the blood vessel from which the corresponding vascular smooth muscle cells originate.

For example, the first type and the second type of vascular smooth muscle cells may originate from blood vessels selected from the aorta, an artery (e.g., a coronary artery or a pulmonary artery), an arteriole, a vein (e.g., a cardiac vein or a pulmonary vein), and a venule of a mammal, preferably of a human, provided that the blood vessels from which the first and the second type of vascular smooth muscle cells originate have a different diameter, as described above. Accordingly, the first type of vascular smooth muscle cells may originate from the human aorta, and the second type of vascular smooth muscle cells may originate from human coronary artery. Alternatively, the first type of vascular smooth muscle cells may originate from a human artery, and the second type of vascular smooth muscle cells may originate from a human arteriole. As a further alternative, the first type of vascular smooth muscle cells may originate from a human vein, and the second type of vascular smooth muscle cells may originate from a human venule. It is particularly preferred that the first type of vascular smooth muscle cells originates from the human aorta, and the second type of vascular smooth muscle cells originates from a human coronary artery. Accordingly, it is particularly preferred that the first type of vascular smooth muscle cells are human aortic smooth muscle cells, and that the second type of vascular smooth muscle cells are human coronary artery smooth muscle cells. In particular, it has been found that cells from the coronary artery react more sensitive, e.g., to a higher phosphate concentration (as calcification inducer) and mineralize much stronger and earlier than cells from the aorta.

Vascular calcification can be induced in the first type and the second type of vascular smooth muscle cells, e.g., by the addition of phosphate (e.g., orthophosphate, hydrogen phosphate or dihydrogen phosphate), β-glycerophosphate, a bone morphogenic protein, and/or cholesterol, by serum depletion, and/or by microcurrent stimulation. The use of phosphate (e.g., orthophosphate) for the induction of calcification is particularly advantageous. For example, phosphate (such as orthophosphate) can be added to the culture media of the first type and the second type of vascular smooth muscle cells to a final concentration in each culture medium of about 1 mM to about 10 mM, preferably about 2 mM to about 5 mM, and more preferably about 3 mM, in order to induce calcification. The first type and the second type of vascular smooth muscle cells may be cultured, e.g., for a period of 3 days to 7 days starting from the induction of calcification, or for a period of 7 days to 14 days starting from the induction of calcification. While calcification can be induced in the course of an experiment using the cell culture model according to the invention, calcified vascular smooth muscle cells, i.e. cells in which calcification has already been induced (e.g., shortly before an intended experiment), can also be employed as the first type and the second type of vascular smooth muscle cells in accordance with the present invention.

In the cell culture model of the invention, the extent of calcification can be determined rapidly and quantitatively by detecting the formation of hydroxyapatite in the first type and the second type of vascular smooth muscle cells. This can be done, e.g., by using histochemical staining (such as Alizarin Red S staining or von Kossa staining), immunohistochemical staining, or optical imaging with a contrast dye such as Cy-HABP-19 (see, e.g., Lee J S et al., Chembiochem. 12(11):1669-73, 2011 or Lee J S et al., Atherosclerosis, 224(2):340-7, 2012). Moreover, the intracellular calcium concentration in the first type and in the second type of vascular smooth muscle cells can be detected, e.g., using a fluorometric calcium assay, with a dye such as Fluo-8, Fluo-8 AM, Rhod-4, or Fura-2.

The cell culture model according to the invention can be put to a variety of different uses, e.g., for investigating vascular calcification processes (particularly beginning (early) vascular calcification processes, including physiological and pathological vascular calcification processes), as well as investigating the induction, inhibition and/or reversion of such calcification processes. In particular, the present invention relates to the use of the cell culture model provided herein (i) for analyzing or examining vascular calcification (particularly beginning/early vascular calcification), (ii) for analyzing the onset and/or the progression of vascular calcification, (iii) for analyzing or examining the reversibility of vascular calcification processes (particularly beginning vascular calcification processes), (iv) for identifying a calcification inhibitor, (v) in a screening method for identifying a calcification inhibitor, (vi) for testing a compound for its suitability as a calcification inhibitor, or (vii) for analyzing the effectiveness and/or mode of action of a calcification inhibitor.

Any of the known calcification inhibitors can thus be examined using the cell culture model according to the present invention, e.g., with respect to their mode of action or their effectiveness on a particular type of blood vessel. Such known calcification inhibitors include, in particular, bisphosphonates (e.g., etidronate, clodronate, tiludronate, pamidronate, neridronate, olpadronate, alendronate, ibandronate, risedronate, zoledronate, incadronate, minodronate, cimadronate, or EB-1053 (i.e., 1-hydroxy-3-(1-pyrrolidinyl)-propylidene-1,1-bisphosphonate)), prednisolone, calcitriol, adenosine triphosphate (ATP) (particularly exogenously administered ATP), fibroblast growth factor 23 (FGF23), Klotho (EC number 3.2.1.31), foscarnet (i.e., phosphonoformic acid), microRNA 205 (miR-205; particularly human miR-205), as well as pharmaceutically acceptable salts and solvates of any of these agents.

Moreover, compounds that are not yet known to inhibit vascular calcification can also be tested for their suitability as calcification inhibitors. Such test compounds are not particularly limited and may, e.g., be selected from small molecules, peptides, proteins, and antibodies. The cell culture model according to the invention thereby allows to identify novel calcification inhibitors, which are also a subject of the present invention.

In accordance with the uses of the culture model described above, the invention also provides a method of analyzing vascular calcification, the method comprising:

culturing a first type of vascular smooth muscle cells and a second type of vascular smooth muscle cells, wherein the first type and the second type of vascular smooth muscle cells originate from blood vessels having a different diameter;

inducing calcification in the first type and in the second type of vascular smooth muscle cells; and

determining the extent of calcification in the first type and in the second type of vascular smooth muscle cells.

The invention further relates to a method of identifying a calcification inhibitor, the method comprising:

culturing a first type of vascular smooth muscle cells and a second type of vascular smooth muscle cells, wherein the first type and the second type of vascular smooth muscle cells originate from blood vessels having a different diameter;

inducing calcification in the first type and in the second type of vascular smooth muscle cells;

adding a test agent to the first type and to the second type of vascular smooth muscle cells;

determining the extent of calcification in the first type and in the second type of vascular smooth muscle cells, both in the presence and in the absence of the test agent; and

identifying the test agent as a calcification inhibitor if the extent of calcification in the first type and/or in the second type of vascular smooth muscle cells is lower in the presence of the test agent than in the absence of the test agent.

The invention likewise relates to a method of determining the suitability of a test agent as a calcification inhibitor, the method comprising:

culturing a first type of vascular smooth muscle cells and a second type of vascular smooth muscle cells, wherein the first type and the second type of vascular smooth muscle cells originate from blood vessels having a different diameter;

inducing calcification in the first type and in the second type of vascular smooth muscle cells;

adding the test agent to the first type and to the second type of vascular smooth muscle cells;

determining the extent of calcification in the first type and in the second type of vascular smooth muscle cells, both in the presence and in the absence of the test agent; and

identifying the test agent as a calcification inhibitor if the extent of calcification in the first type and/or in the second type of vascular smooth muscle cells is lower in the presence of the test agent than in the absence of the test agent.

Moreover, the invention also provides a method of analyzing the effectiveness of a calcification inhibitor, the method comprising:

culturing a first type of vascular smooth muscle cells and a second type of vascular smooth muscle cells, wherein the first type and the second type of vascular smooth muscle cells originate from blood vessels having a different diameter;

inducing calcification in the first type and in the second type of vascular smooth muscle cells;

adding the calcification inhibitor to the first type and to the second type of vascular smooth muscle cells; and

determining the extent of calcification in the first type and in the second type of vascular smooth muscle cells.

The present invention furthermore relates to the use of the cell culture model provided herein for analyzing the effect of electrical stimulation, preferably of microcurrent stimulation, on vascular calcification. Such electrical stimulation (or microcurrent stimulation) can be effected, in particular, by applying an electrical current of about 0.1 μA to about 100 μA at a frequency of about 1 mHz to about 25 Hz, preferably about 0.5 μA to about 20 μA at a frequency of about 1 mHz to about 25 Hz, to the first type and the second type of cultured vascular smooth muscle cells.

The cell culture model according to the invention thus allows to test the effect of an electrical current, such as a microcurrent, applied to a “biological system” like calcified vascular cells in an advantageously simple experimental setting. In accordance with the invention, it is contemplated that the electrical current modifies calcification processes and gene expression of biomarkers and, in turn, eventually improves the function of the calcified blood vessels concomitantly with a clinical benefit, which may be used for a novel treatment of vascular calcification processes.

Accordingly, the invention also relates to a method of analyzing the effect of electrical stimulation on vascular calcification, the method comprising:

culturing a first type of vascular smooth muscle cells and a second type of vascular smooth muscle cells, wherein the first type and the second type of vascular smooth muscle cells originate from blood vessels having a different diameter;

inducing calcification in the first type and in the second type of vascular smooth muscle cells;

applying an electrical current, preferably a microcurrent, to the first type and to the second type of vascular smooth muscle cells; and

determining the extent of calcification in the first type and in the second type of vascular smooth muscle cells.

The invention further relates to the use of the cell culture model provided herein for identifying or verifying a biomarker of vascular calcification, or for analyzing the expression of a biomarker of vascular calcification (e.g., for analyzing the temporal expression pattern of a biomarker of vascular calcification). Such a biomarker of vascular calcification is preferably an expression product of a marker gene (e.g., in the form of a nucleic acid, such as mRNA, or in the form of a protein). Examples of known biomarkers of vascular calcification include, in particular, osteoprotegerin (OPG), osteopontin (OPN), osteocalcin (OC), osterix (OSX), matrix gla-protein (MGP), fetuin A, alkaline phosphatase (AP), core-binding factor alpha 1 (Cbfa-1), fibroblast growth factor 23 (FGF-23), sclerostin (SOST), osteonectin (SPARC), Klotho (KL), receptor activator of nuclear factor κ-B ligand (RANKL), stanniocalcin-1 (STC1), stanniocalcin-2 (STC2), or Dickkopf-related protein 1 (DKK1). However, the cell culture model of the present invention also allows to identify and/or verify novel biomarkers of vascular calcification, e.g., by analyzing the expression of a potential biomarker and correlating its level of expression or the change in its level of expression with the beginning, progression, halt or reversal of vascular calcification. The identification and/or verification of a biomarker may comprise, e.g., a step of determining the level of expression of the biomarker after induction of calcification and a step of comparing said level of expression to a reference expression level (e.g., a reference expression level of the same biomarker in the absence of calcification). The expression of a biomarker may be analyzed by analyzing its transcription, e.g., using a quantitative reverse transcriptase polymerase chain reaction (qRT-PCR) or a microarray. The expression of a biomarker may also be analyzed by analyzing its translation, e.g., using an antibody-based assay, mass spectrometry, a gel-based or blot-based assay, or flow cytometry, preferably using an antibody-based assay selected from an immunohistochemical method, an enzyme-linked immunosorbent assay, and a radioimmunoassay.

Thus, using the cell culture model provided herein, the present invention allows to (i) determine the beginning of calcification in human vascular smooth muscle cells in vitro, (ii) depict the progress of calcification in human vascular smooth muscle cells isolated from vessels of different size (e.g., aorta and coronary artery), (iii) determine the temporal expression of known calcification biomarkers, (iv) examine the use of calcification inhibitors (e.g., bisphosphonates) in vitro, (v) determine the effect of different calcification inhibitor (e.g., bisphosphonate) concentrations on calcified vascular smooth muscle cells, including determining a halt in the progression of calcification or a reversal of calcification, (vi) examine the application of electrical currents (particularly microcurrents) to calcified vascular smooth muscle cells, (vii) determine the effect of microcurrents on calcified vascular smooth muscle cells, including determining a halt in the progression of calcification or a reversal of calcification, and/or (viii) determine the expression of known calcification biomarkers.

As used herein, the term “small molecule” refers to any molecule, particularly any organic molecule (i.e., any molecule containing, inter alia, carbon atoms), that has a molecular weight of equal to or less than about 900 Da, preferably of equal to or less than about 500 Da. The molecular weight of a molecule can be determined using methods known in the art, such as, e.g., mass spectrometry (e.g., electrospray ionization mass spectrometry (ESI-MS) or matrix-assisted laser desorption/ionization mass spectrometry (MALDI-MS)), gel electrophoresis (e.g., polyacrylamide gel electrophoresis using sodium dodecyl sulfate (SDS-PAGE)), hydrodynamic methods (e.g., gel filtration chromatography or gradient sedimentation), or static light scattering (e.g., multi-angle light scattering (MALS)), and is preferably determined using mass spectrometry.

The terms “peptide” and “protein” are used herein interchangeably and refer to a polymer of two or more amino acids linked via amide bonds that are formed between an amino group of one amino acid and a carboxyl group of another amino acid. The amino acids comprised in the peptide or protein, which are also referred to as amino acid residues, may be selected from the 20 standard proteinogenic α-amino acids (i.e., Ala, Arg, Asn, Asp, Cys, Glu, Gln, Gly, His, Ile, Leu, Lys, Met, Phe, Pro, Ser, Thr, Trp, Tyr, and Val) but also from non-proteinogenic and/or non-standard α-amino acids (such as, e.g., ornithine, citrulline, homolysine, pyrrolysine, or 4-hydroxyproline) as well as β-amino acids (e.g., β-alanine), γ-amino acids and δ-amino acids. Preferably, the amino acid residues comprised in the peptide or protein are selected from α-amino acids, more preferably from the 20 standard proteinogenic α-amino acids (which can be present as the L-isomer or the D-isomer, and are preferably all present as the L-isomer). The peptide or protein may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts P G & Greene T W, Greene's protective groups in organic synthesis, John Wiley & Sons, 2006. Such modifications may also include the covalent attachment of one or more polyethylene glycol (PEG) chains (forming a PEGylated peptide or protein), the glycosylation and/or the acylation with one or more fatty acids (e.g., one or more C₈₋₃₀ alkanoic or alkenoic acids; forming a fatty acid acylated peptide or protein). The amino acid residues comprised in the peptide or protein may, e.g., be present as a linear molecular chain (forming a linear peptide or protein) or may form one or more rings (corresponding to a cyclic peptide or protein). The peptide or protein may also form oligomers consisting of two or more identical or different molecules.

The term “in vitro” is used herein in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The term “about”, as used herein, preferably refers to ±10% of the indicated numerical value, more preferably to ±5% of the indicated numerical value, and in particular to the exact numerical value indicated. For example, the expression “about 100” preferably refers to 100±10% (i.e., 90 to 110), more preferably to 100±5% (i.e., 95 to 105), and even more preferably to the specific value of 100. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint.

The term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements/components/steps (e.g., “A containing B, C and D” would also be encompassed), but this term also includes the meaning of “A consisting essentially of B and C” and the meaning of “A consisting of B and C” (i.e., no other elements/components/steps than B and C are comprised in A).

The different method steps of the methods described herein can, in general, be carried out in any suitable order, unless indicated otherwise or contradicted by context, and are preferably carried out in the specific order in which they are indicated.

It is to be understood that the present invention specifically relates to each and every combination of features and embodiments described herein, including any combination of general and/or preferred features/embodiments.

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention is also described by the following illustrative figures. The appended figures show:

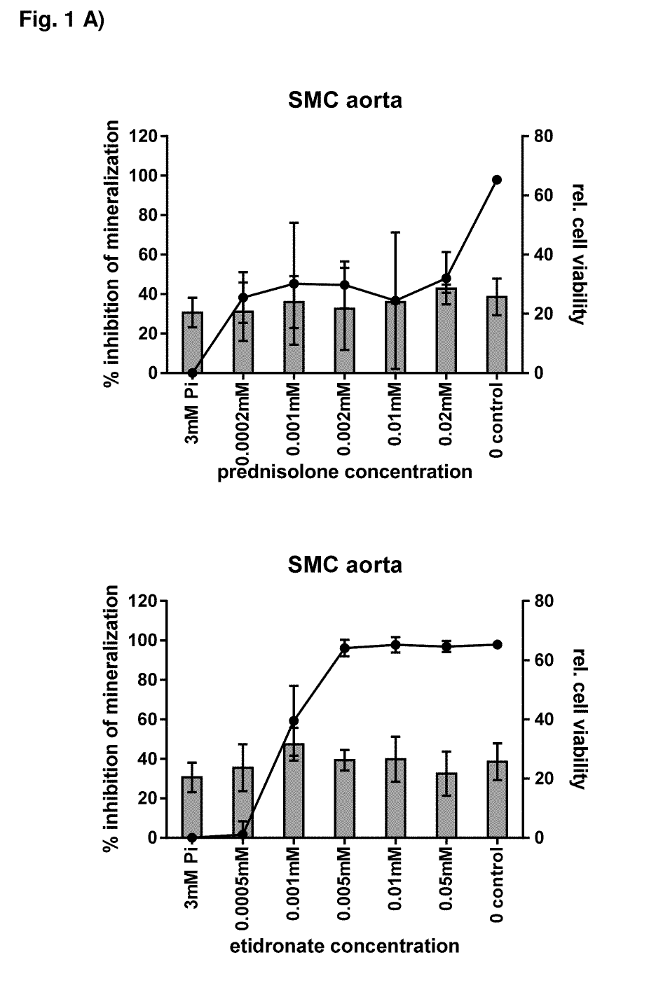

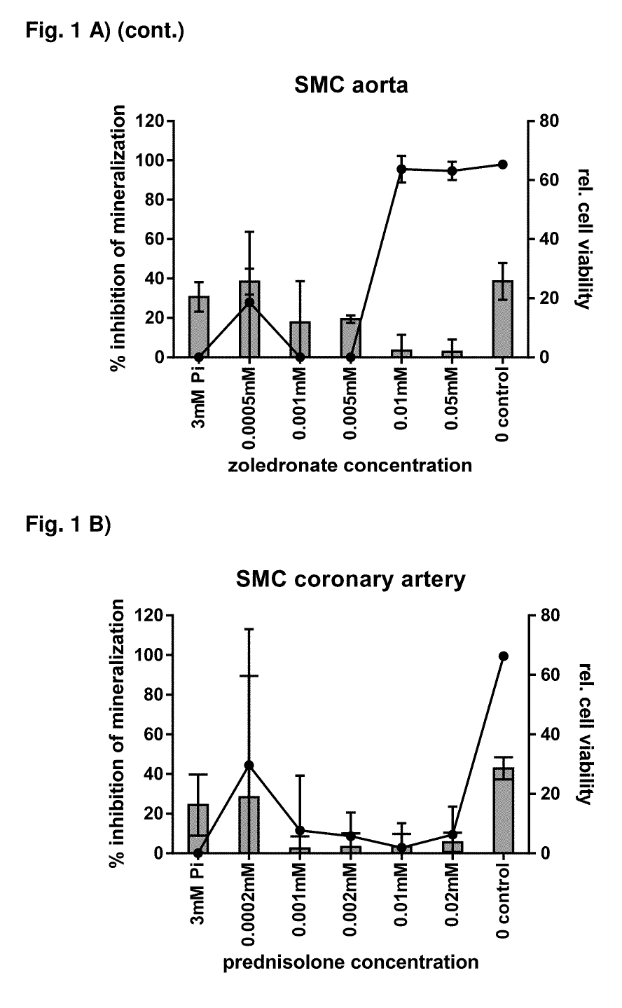

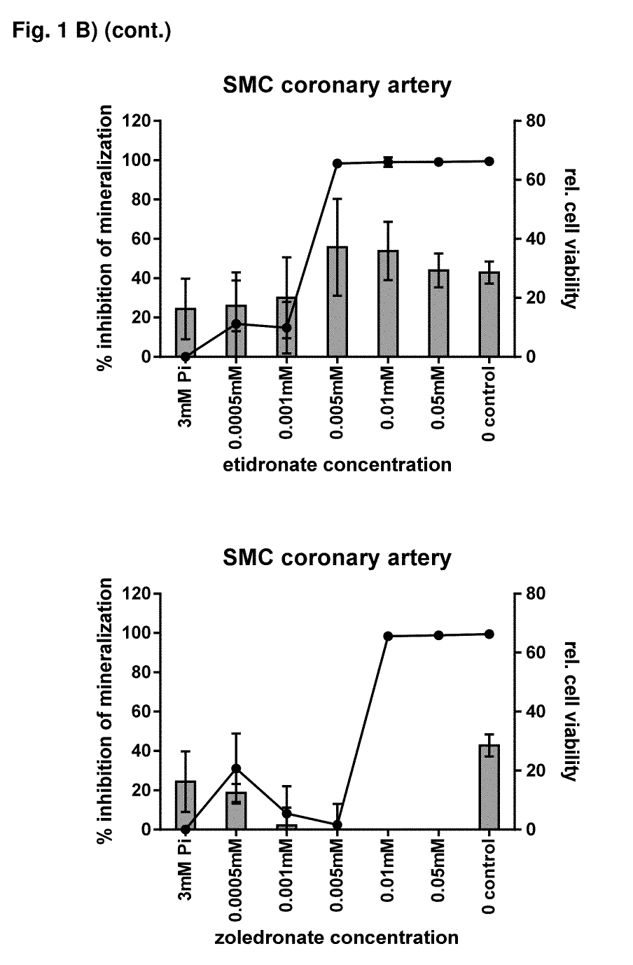

FIG. 1: Calcification of vascular smooth muscle cells was induced by a phosphate medium (3 mM sodium phosphate) on culture day 0, and 1.5 mM sodium phosphate was added to the medium on culture day 2, 4 and 6 additionally. Prednisolone, etidronate and zoledronate were added in different concentrations on culture day 0, 2, 4 and 6. Hydroxy apatite formation was determined by using a mineralization assay (Lonza). Calcification was normalized relative to the total protein concentration (Bradford assay). (A) Effect of substances on calcified human aortic smooth muscle cells on culture day 7. (B) Effect of substances on calcified human coronary artery smooth muscle cells on culture day 7. Mineralization is shown as a line, cell viability as bars.

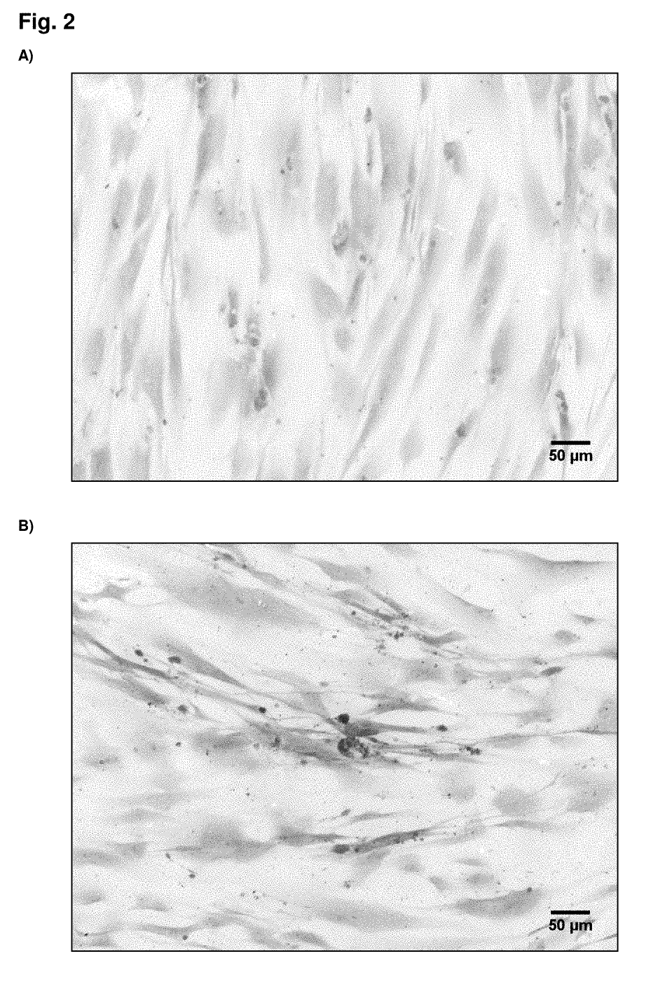

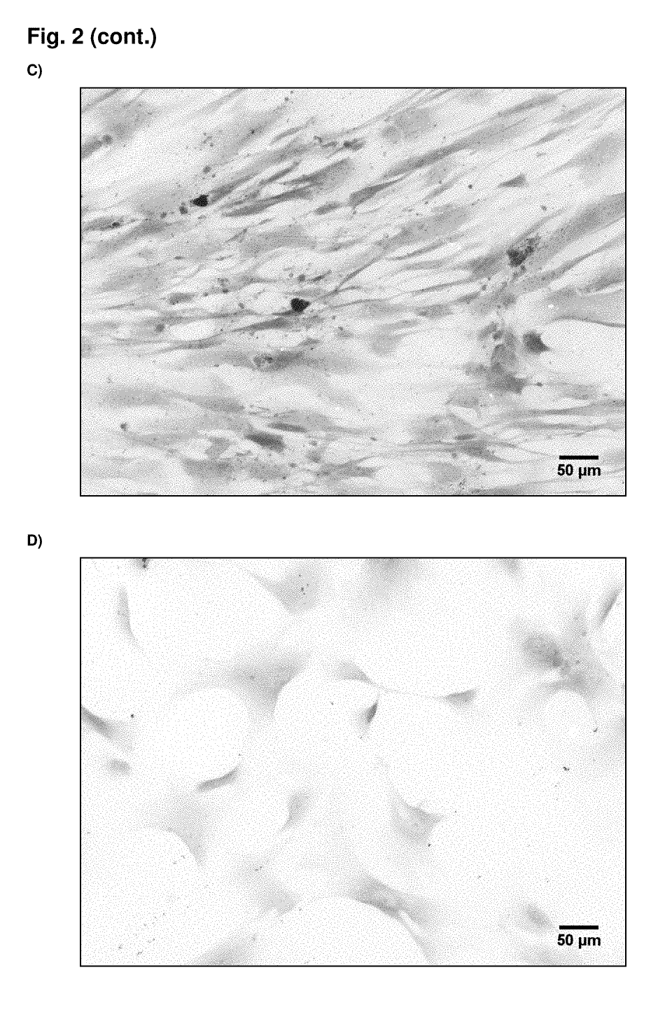

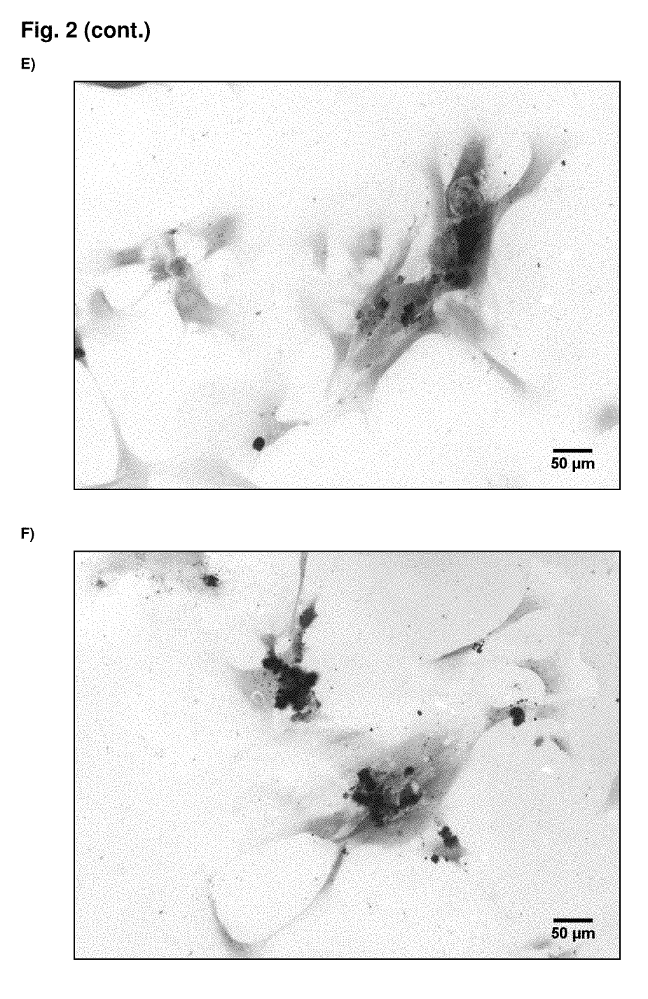

FIG. 2: Von Kossa staining (200× magnification) of human vascular smooth muscle cells of the aorta (A-C) and coronary artery (D-F). Staining on culture day 1 (A, D), day 2 (B, E) and day 7 (C, F). Black spots indicate mineralization.

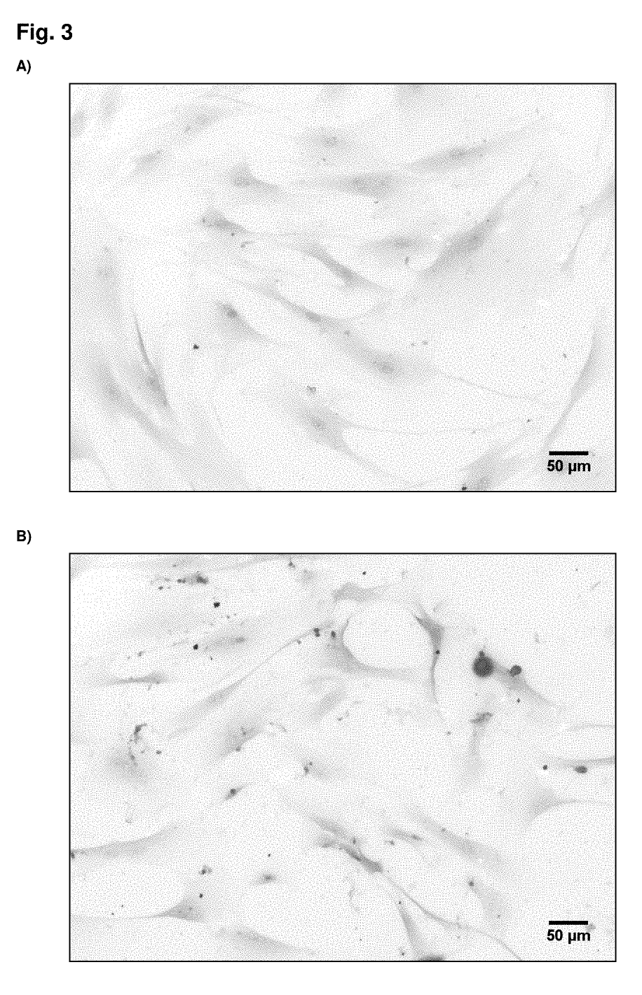

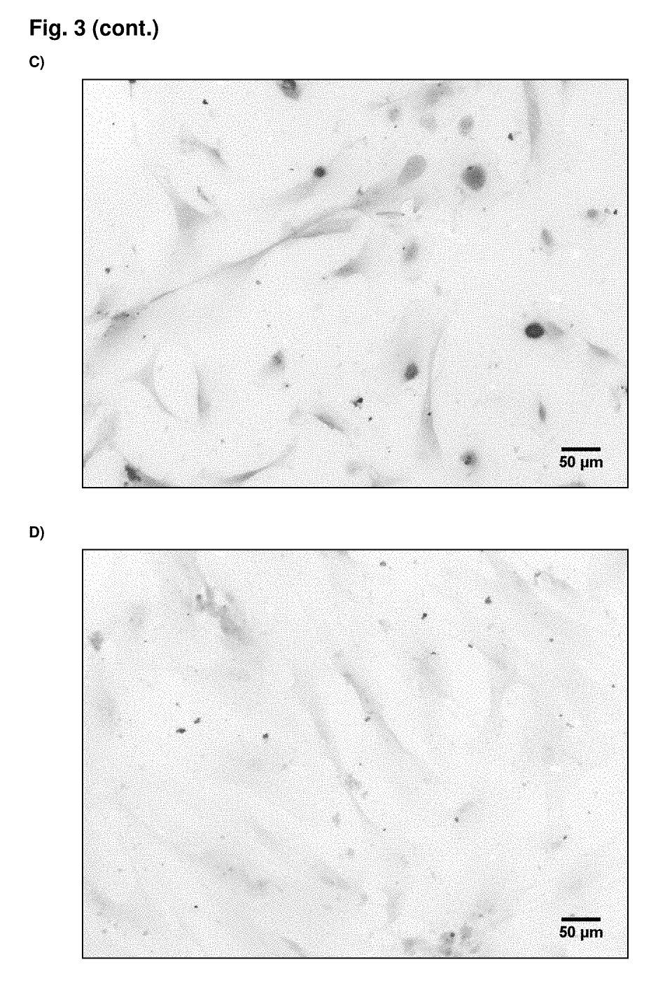

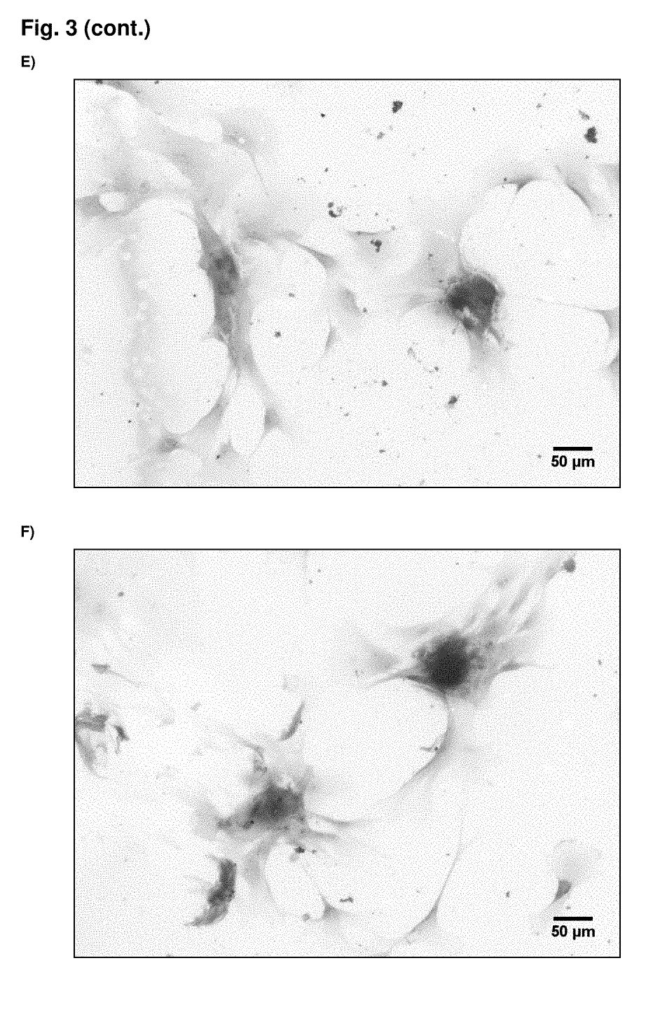

FIG. 3: Alizarin Red S staining (200× magnification) of human vascular smooth muscle cells of the aorta (A-C) and coronary artery (D-F). Staining on culture day 1 (A, D), day 2 (B, E) and day 7 (C, F). Black spots indicate mineralization.

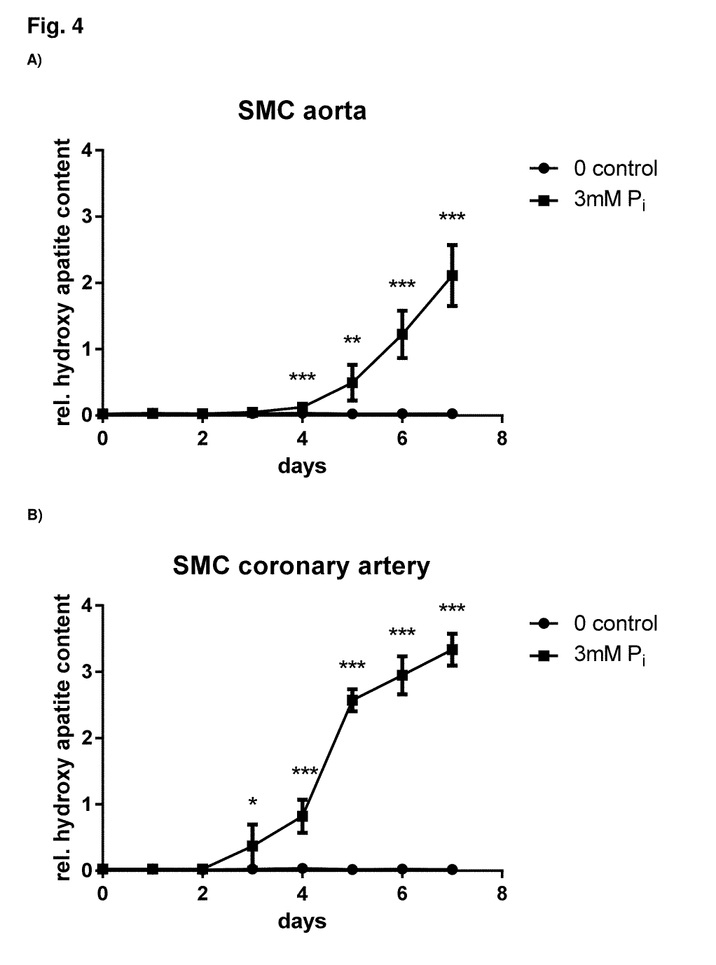

FIG. 4: Hydroxy apatite formation in human vascular smooth muscle cells of the aorta (A) and coronary artery (B) after and without addition of phosphate. Values are normalized via total protein content.

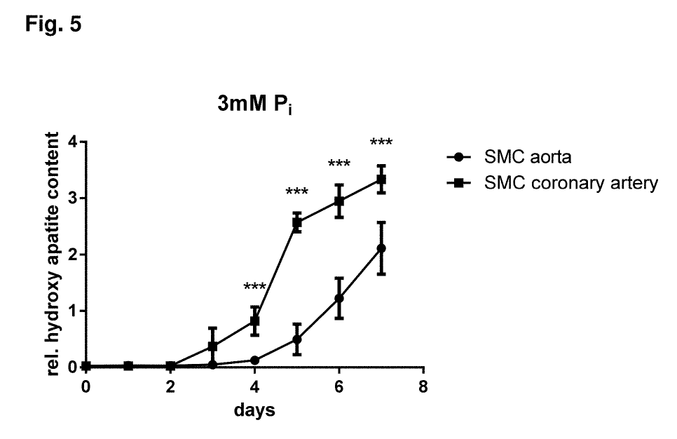

FIG. 5: Comparison of the degree of calcification between human vascular smooth muscles cells of the aorta and coronary artery. Values are normalized via total protein content.

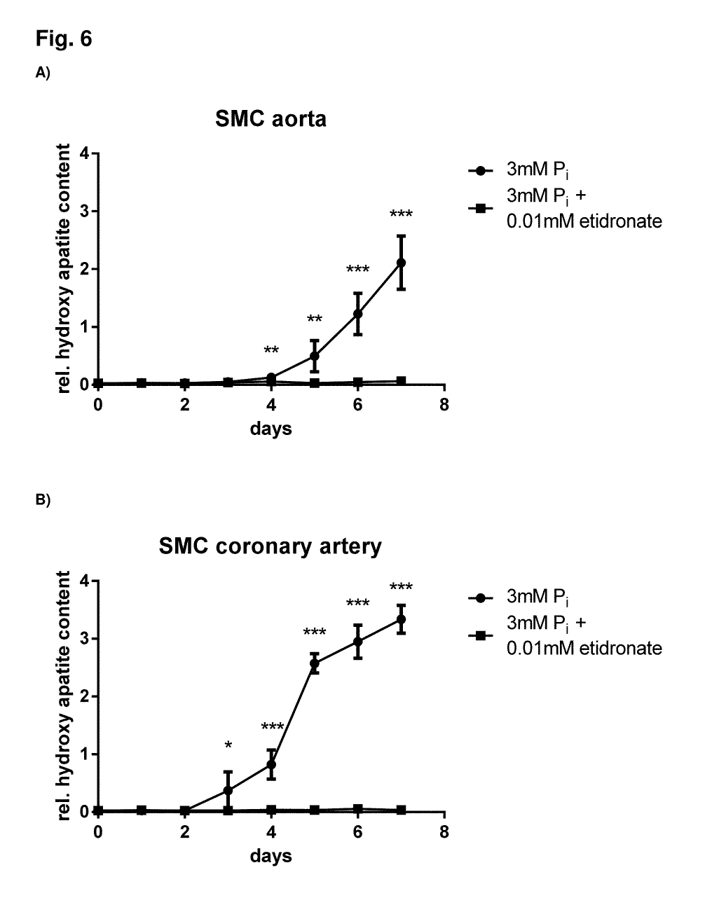

FIG. 6: Mineralization process in human vascular smooth muscle cells of the aorta (A) and coronary artery (B) with the addition of phosphate and 0.01 mM etidronate. Values are normalized via total protein content.

FIG. 7: Mineralization process in human vascular smooth muscle cells of the aorta (A) and coronary artery (B) with the addition of phosphate and 0.001 mM etidronate. Values are normalized via total protein content.

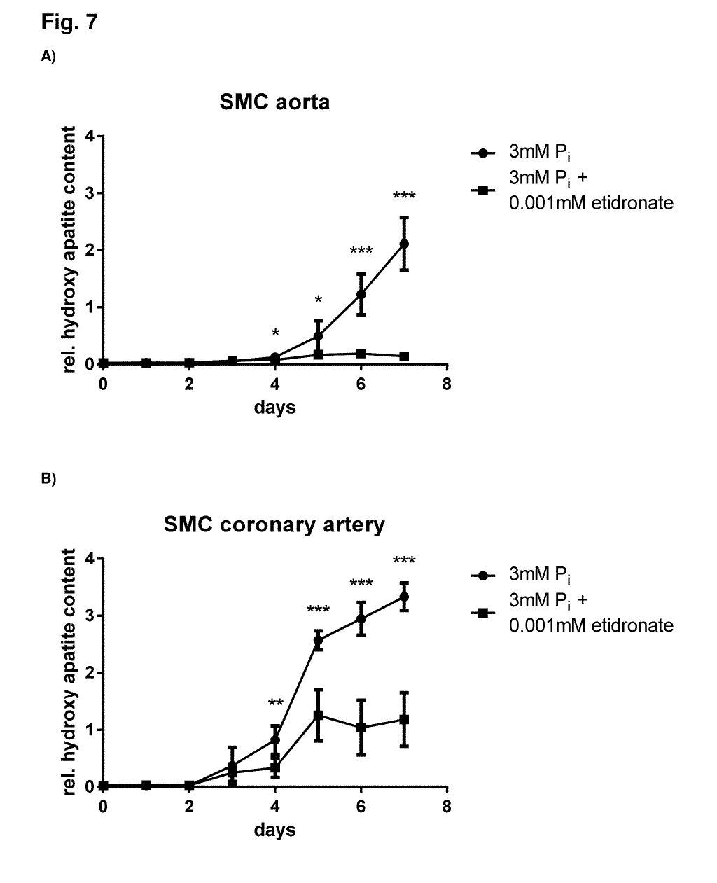

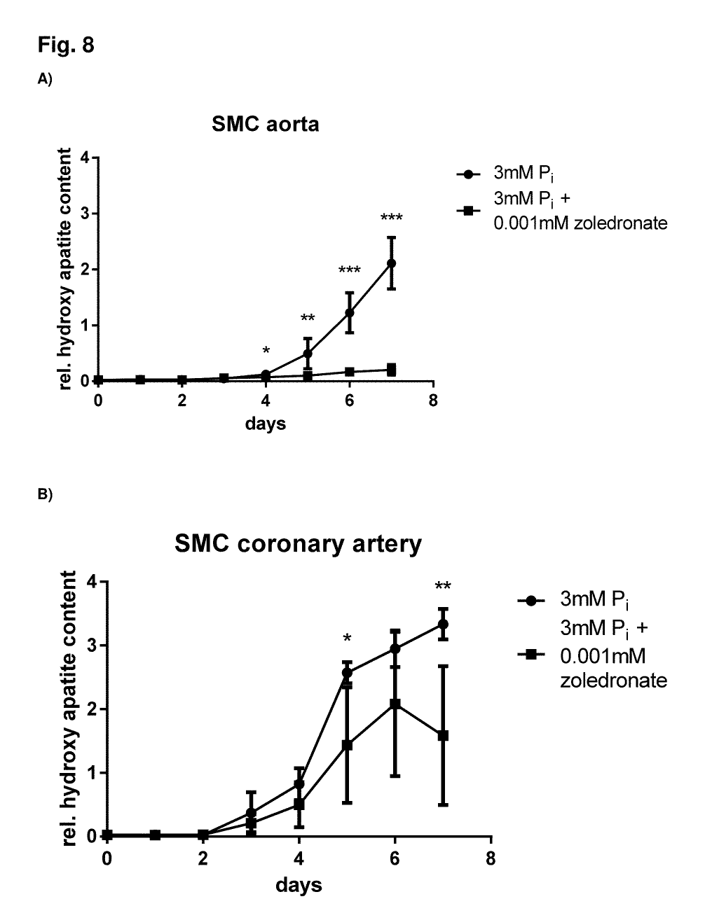

FIG. 8: Mineralization process in human vascular smooth muscle cells of the aorta (A) and coronary artery (B) with the addition of phosphate and 0.001 mM zoledronate. Values are normalized via total protein content.

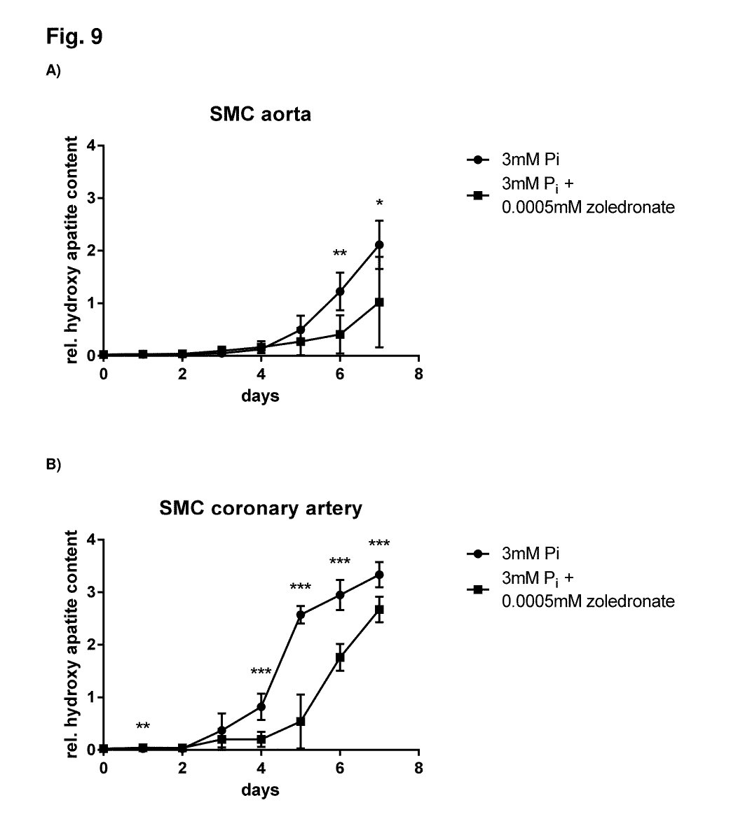

FIG. 9: Mineralization process in human vascular smooth muscle cells of the aorta (A) and coronary artery (B) with the addition of phosphate and 0.0005 mM zoledronate. Values are normalized via total protein content.

FIG. 10: Mineralization process in human vascular smooth muscle cells of the aorta (A) and coronary artery (B) with the addition of phosphate and 0.02 mM prednisolone. Values are normalized via total protein content.

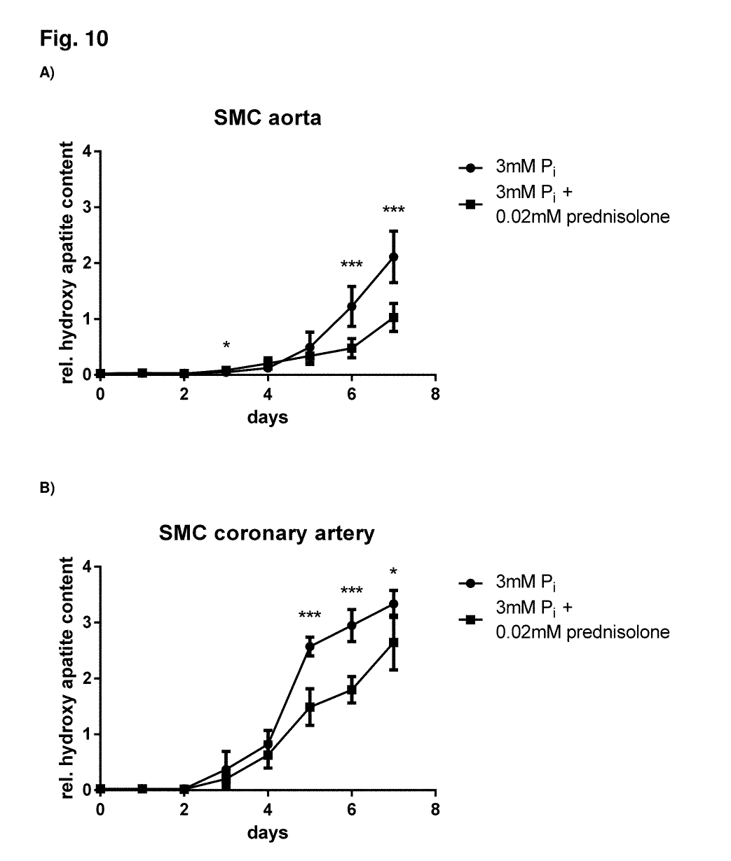

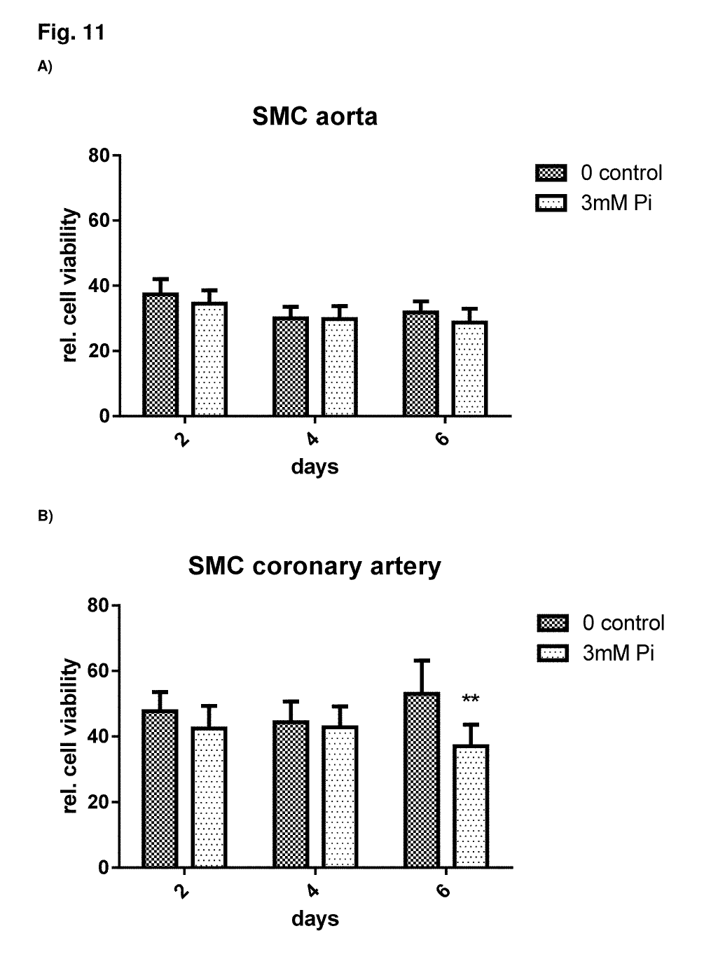

FIG. 11: Cell viability of human vascular smooth muscle cells of the aorta (A) and the coronary artery (B) after and without addition of phosphate. Values are normalized via total protein content.

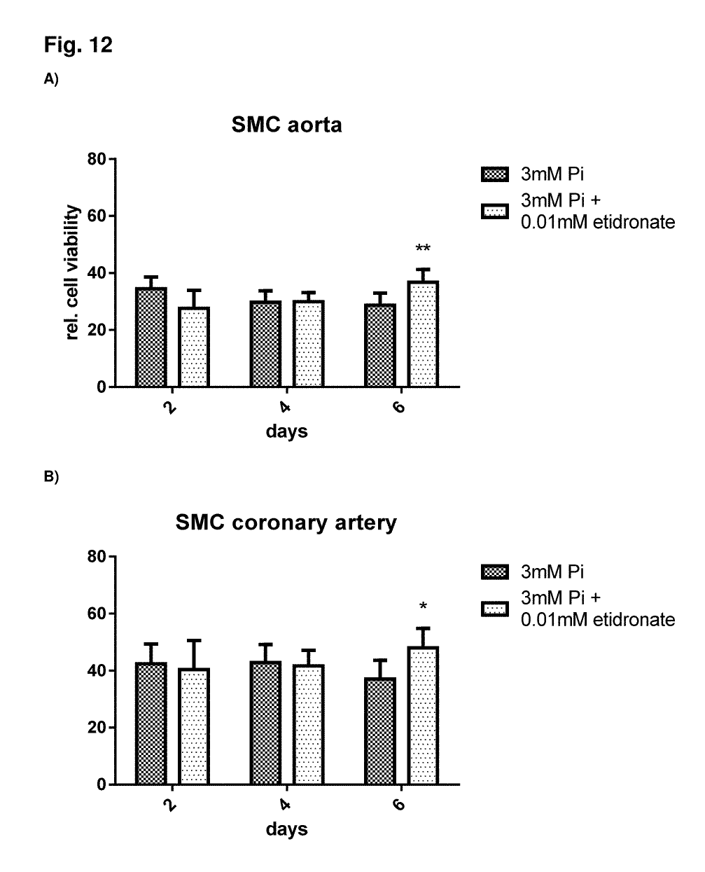

FIG. 12: Cell viability of human vascular smooth muscle cells of the aorta (A) and the coronary artery (B) after and without addition of 0.01 mM etidronate. Values are normalized via total protein content.

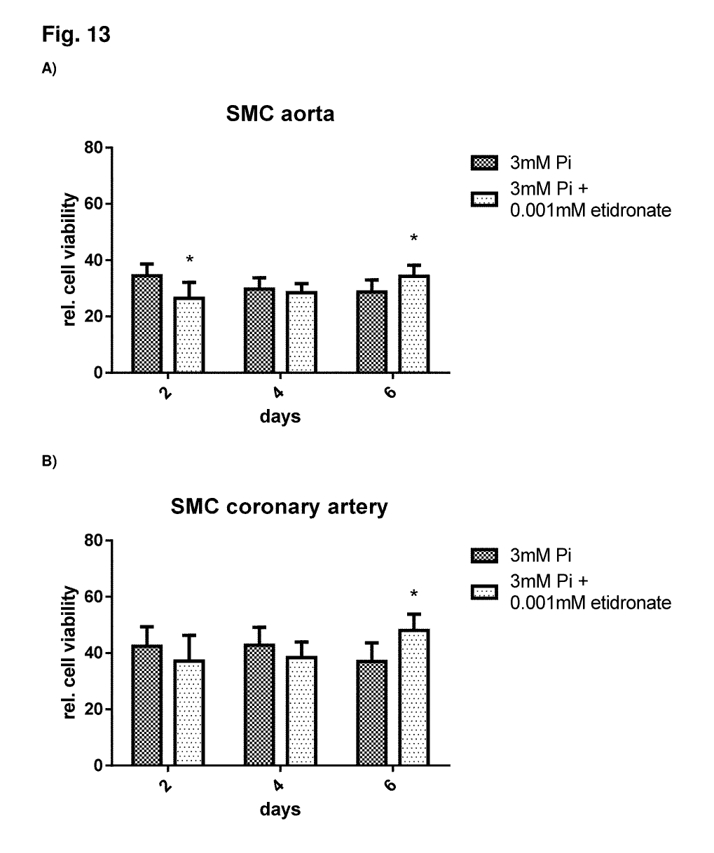

FIG. 13: Cell viability of human vascular smooth muscle cells of the aorta (A) and the coronary artery (B) after and without addition of 0.001 mM etidronate. Values are normalized via total protein content.

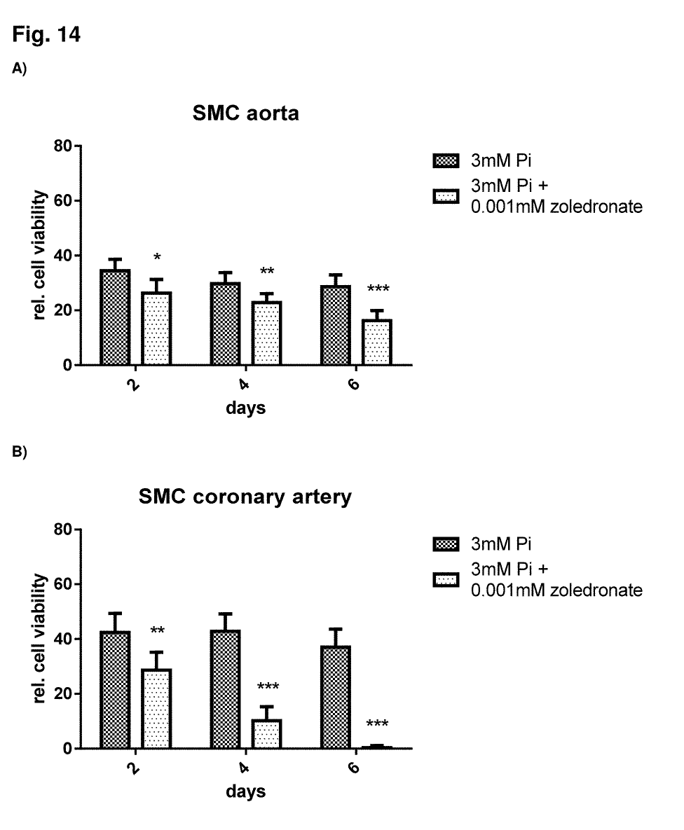

FIG. 14: Cell viability of human vascular smooth muscle cells of the aorta (A) and the coronary artery (B) after and without addition of 0.001 mM zoledronate. Values are normalized via total protein content.

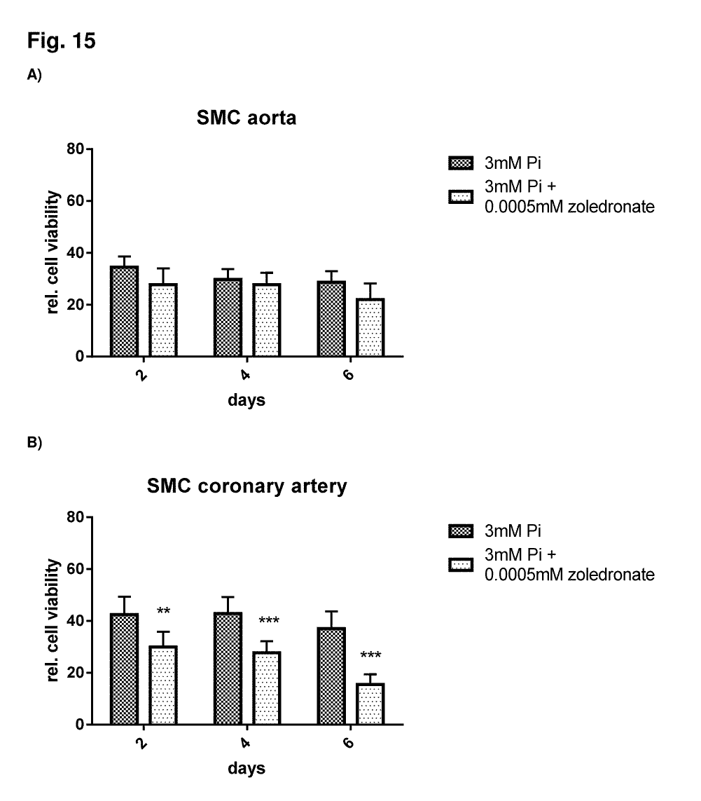

FIG. 15: Cell viability of human vascular smooth muscle cells of the aorta (A) and the coronary artery (B) after and without addition of 0.0005 mM zoledronate. Values are normalized via total protein content.

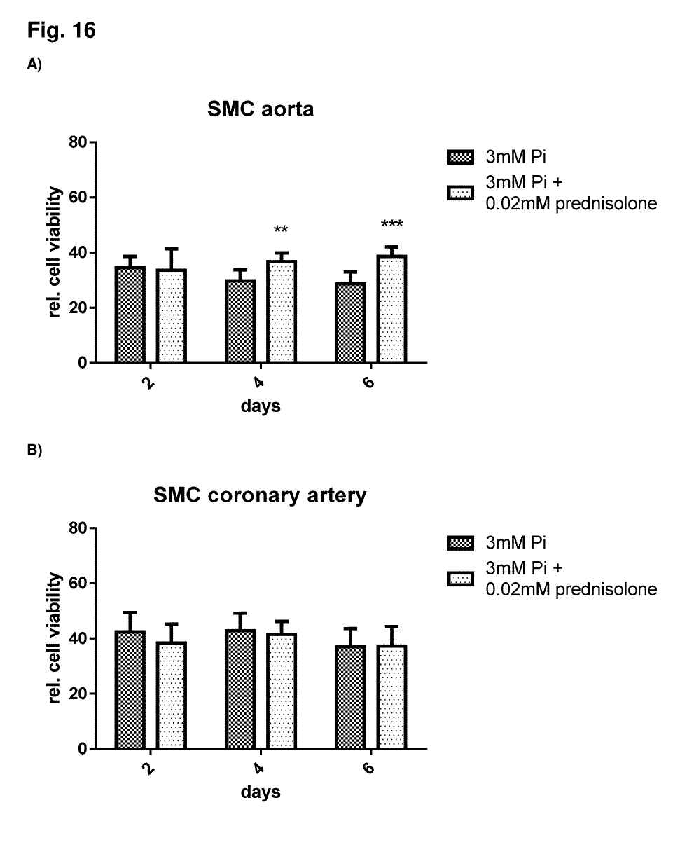

FIG. 16: Cell viability of human vascular smooth muscle cells of the aorta (A) and the coronary artery (B) after and without addition of 0.02 mM prednisolone. Values are normalized via total protein content.

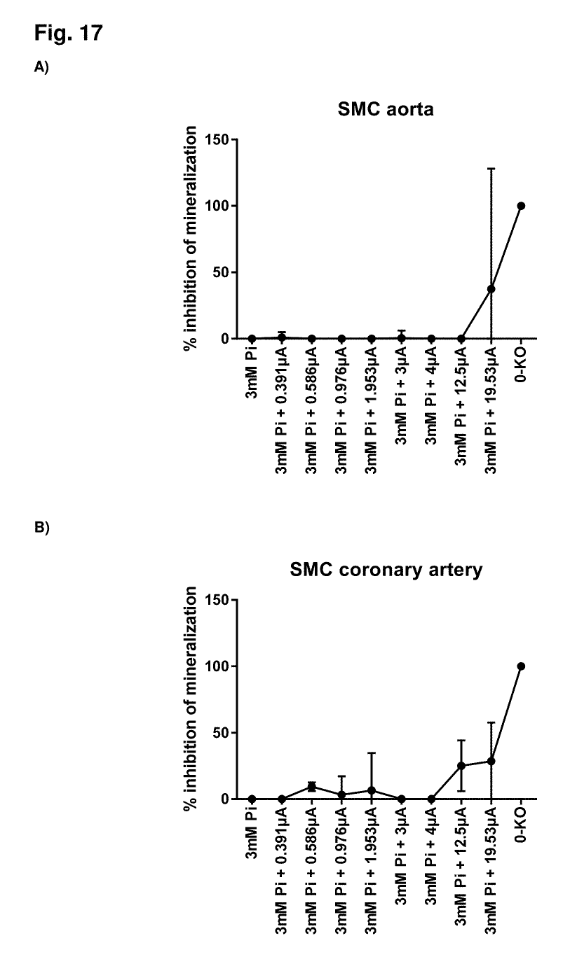

FIG. 17: Microcurrent application with 19.53 μA on vascular smooth muscle cells of the aorta (A) and coronary artery (B) from culture day 0 till day 7. Values are normalized via total protein content.

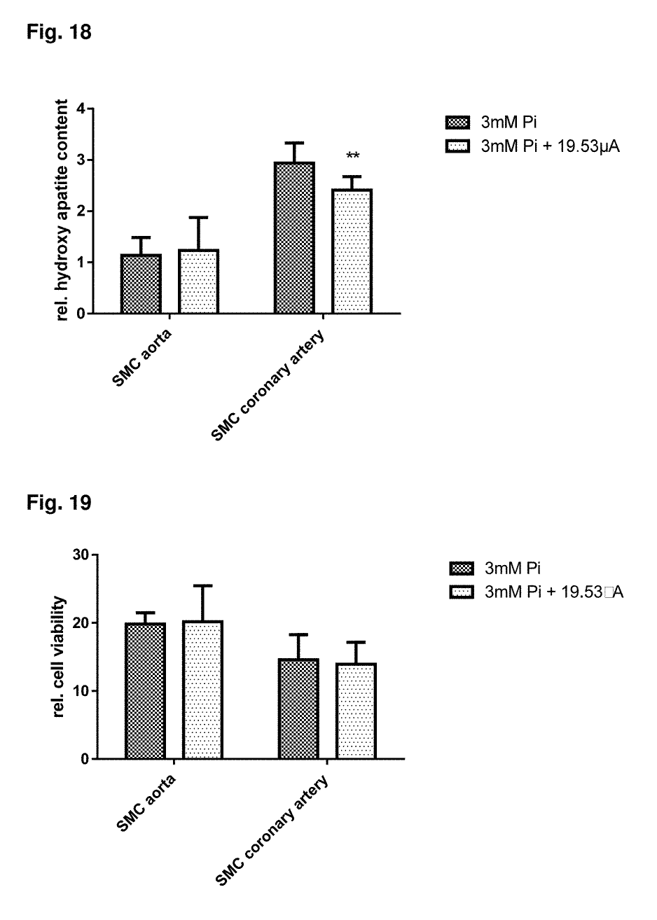

FIG. 18: Microcurrent application with 19.53 μA on vascular smooth muscle cells of the aorta and coronary artery from culture day 0 till day 7. Values are normalized via total protein content.

FIG. 19: Cell viability of human vascular smooth muscle cells of the aorta and the coronary artery after microcurrent application with 19.53 μA. Values are normalized via total protein content.

The invention will now be described by reference to the following examples which are merely illustrative and are not to be construed as a limitation of the scope of the present invention.

### EXAMPLES

### Example 1

### Vascular Calcification in Primary Human Vascular Smooth Muscle Cells Originated from Aorta and Coronary Artery, Respectively

Calcification in primary human vascular smooth muscle cells was induced in vitro by the addition of 3 mM sodium phosphate into culture medium (Dulbecco's modified eagle's medium containing 10% fetal calf serum, 1% penicillin streptomycin, 1% non essential amino acids, 1% ascorbic acid, 1% transferrin, 1% sodium selenite, 0.1% endothelial cell growth supplement and 0.1% insulin). Cell cultivation was performed up to 7 days. To differentiate calcification processes, smooth muscle cells originated from vessels of different size (aorta and ae. coronariae) were analysed. Human cells were purchased as primary cells to ensure highest quality.

24 well clear tissue culture-treated plates were prepared for use. Human vascular smooth muscle cells were trypsinised by using Trypsin/Ethylenediaminetetraacetic acid and the cell suspension was centrifuged at 1200 rpm for 8 minutes. Thereafter cells were resuspended in medium, cell number was determined and the cell density was defined by 3×10⁴ cells in 500 μL culture medium per well. Cells were incubated at +37° C. and 5% CO₂ over night. Afterwards the culture medium was replaced by 1 mL of high phosphate medium. Every second day 1/100 of the medium was replaced by fresh phosphate medium (150 mM) as well.

For testing of bisphosphonates and prednisolone 1/100 of the medium per well was replaced by the particular concentration of these substances on culture day 0, 2, 4 and 6.

Additionally, cells were stimulated electrically by use of a direct current power generator via two electrodes. The electrodes were integrated into the top cover of the culture plate and connected to the microcurrent generator. The cells were either left unstimulated or stimulated with microcurrent over a period of 7 days. Every second day 1/100 of the medium was replaced by fresh phosphate medium (150 mM) as well.

Each day a fluorometric mineralization assay (Lonza, Osteolmage) was performed to detect the amount of hydroxy apatite formation. Therefore the medium was removed and cells were washed once with PBS. For fixation cells were incubated with 100% ethanol for 20 minutes. After fixation cells were washed with 1 mL wash buffer and incubated with 500 μL of the staining reagent for 30 minutes. After incubation step the reagent was removed and cells were washed 3 times with 1 mL wash buffer, leaving buffer in the wells for 5 minutes per wash. After final wash, 1 mL buffer was added to each well for fluorescent plate reader analysis. Fluorescence was measured at appropriate excitation/emission wavelengths (492 nm/520 nm).

Data was normalized via total protein content (Bradford assay). Therefore cells were washed with PBS and cells were incubated with 200 μL lysis buffer (150 mM sodium chloride, 0.01% Triton X-100, 50 mM Tris pH 8.0) for 20 minutes. Thereafter 600 μL H₂O_{ddest} and 200 μL BioRad protein assay solution were added to lysis buffer. After 10 minutes incubation photometric analysis was performed with a wavelength of 595 nm.

On culture day 2, 4 and 6 a cell viability assay was performed additionally. Therefore a 10% PrestoBlue® solution (in culture medium) was prepared. Medium of each well was replaced by the solution and cells were incubated at +37° C. and 5% CO₂ for 1 hour. Fluorescence was measured at appropriate excitation/emission wavelengths (570 nm/585 nm).

The experimental results obtained are shown in FIGS. 1 to 19.

1. A cell culture model of vascular calcification, comprising a first type of cultured vascular smooth muscle cells and a second type of cultured vascular smooth muscle cells, wherein the first type and the second type of vascular smooth muscle cells originate from blood vessels having a different diameter.

2. The cell culture model of claim 1, wherein the first type and the second type of vascular smooth muscle cells are human cells.

3. The cell culture model of claim 1 or 2, wherein the first type and the second type of vascular smooth muscle cells are primary human vascular smooth muscle cells.

4. The cell culture model of any one of claims 1 to 3, wherein the first type of vascular smooth muscle cells originates from a blood vessel having a diameter that is at least about three times as large as the diameter of the blood vessel from which the second type of vascular smooth muscle cells originates.

5. The cell culture model of any one of claims 1 to 4, wherein the first type of vascular smooth muscle cells originates from a blood vessel having a diameter that is at least about 10 times as large as the diameter of the blood vessel from which the second type of vascular smooth muscle cells originates.

6. The cell culture model of any one of claims 1 to 5, wherein the first type of vascular smooth muscle cells originates from a blood vessel having a diameter that is at least about 50 times as large as the diameter of the blood vessel from which the second type of vascular smooth muscle cells originates.

7. The cell culture model of any one of claims 1 to 6, wherein the first type of vascular smooth muscle cells originates from a blood vessel having a diameter that is at least about 200 times as large as the diameter of the blood vessel from which the second type of vascular smooth muscle cells originates.

8. The cell culture model of any one of claims 1 to 7, wherein the first type of vascular smooth muscle cells originates from human aorta, and wherein the second type of vascular smooth muscle cells originates from human coronary artery.

9. The cell culture model of any one of claims 1 to 7, wherein the first type of vascular smooth muscle cells originates from a human artery, and wherein the second type of vascular smooth muscle cells originates from a human arteriole.

10. The cell culture model of any one of claims 1 to 7, wherein the first type of vascular smooth muscle cells originates from a human vein, and wherein the second type of vascular smooth muscle cells originates from a human venule.

11. The cell culture model of any one of claims 1 to 10, wherein calcification has been induced in the first type and the second type of vascular smooth muscle cells.

12. The cell culture model of claim 11, wherein calcification has been induced by orthophosphate, a bone morphogenic protein, cholesterol, serum depletion, or microcurrent stimulation.

13. The cell culture model of claim 11 or 12, wherein calcification has been induced by orthophosphate.

14. The cell culture model of any one of claims 11 to 13, wherein calcification has been induced by the addition of orthophosphate to the culture media of the first type and the second type of vascular smooth muscle cells to a final concentration of about 1 mM to about 10 mM, preferably about 3 mM, in each culture medium.

15. Use of the cell culture model of any one of claims 1 to 14 for analyzing vascular calcification.

16. Use of the cell culture model of any one of claims 1 to 14 for identifying a calcification inhibitor.

17. Use of the cell culture model of any one of claims 1 to 14 for testing a compound for its suitability as a calcification inhibitor.

18. Use of the cell culture model of any one of claims 1 to 14 for analyzing the effectiveness and/or mode of action of a calcification inhibitor.

19. The use of claim 18, wherein the calcification inhibitor is selected from a bisphosphonate, prednisolone, calcitriol, adenosine triphosphate, fibroblast growth factor 23, Klotho, foscarnet, microRNA 205, and a pharmaceutically acceptable salt or solvate of any of the aforementioned agents.

20. The use of claim 18 or 19, wherein the calcification inhibitor is a bisphosphonate which is preferably selected from etidronate, clodronate, tiludronate, pamidronate, neridronate, olpadronate, alendronate, ibandronate, risedronate, zoledronate, incadronate, minodronate, cimadronate, EB-1053, and a pharmaceutically acceptable salt or solvate thereof.

21. Use of the cell culture model of any one of claims 1 to 14 for analyzing the effect of electrical stimulation, preferably of microcurrent stimulation, on vascular calcification.

22. The use of claim 21, wherein the electrical stimulation is effected by applying an electrical current of about 0.1 μA to about 100 μA, preferably about 0.5 μA to about 20 μA, at a frequency of about 1 mHz to about 25 Hz, to the first type and the second type of cultured vascular smooth muscle cells.

23. Use of the cell culture model of any one of claims 1 to 14 for identifying a biomarker of vascular calcification.

24. The use of claim 23, wherein the biomarker is identified by analyzing the expression of the biomarker.

25. Use of the cell culture model of any one of claims 1 to 14 for analyzing the expression of a biomarker of vascular calcification.

26. The use of claim 24 or 25, wherein the expression of the biomarker is analyzed by analyzing the transcription of the biomarker.

27. The use of claim 26, wherein the transcription of the biomarker is analyzed using a quantitative reverse transcriptase polymerase chain reaction or a microarray.

28. The use of claim 24 or 25, wherein the expression of the biomarker is analyzed by analyzing the translation of the biomarker.

29. The use of claim 28, wherein the translation of the biomarker is analyzed using an antibody-based assay, mass spectrometry, a gel-based or blot-based assay, or flow cytometry.

30. The use of claim 29, wherein the translation of the biomarker is analyzed using an antibody-based assay which is selected from an immunohistochemical method, an enzyme-linked immunosorbent assay, and a radioimmunoassay.

31. The use of claim 25 or any one of its dependent claims 26 to 30, wherein the biomarker of vascular calcification is selected from osteoprotegerin (OPG), osteopontin (OPN), osteocalcin (OC), osterix (OSX), matrix gla-protein (MGP), fetuin A, alkaline phosphatase (AP), core-binding factor alpha 1 (Cbfa-1), fibroblast growth factor 23 (FGF-23), sclerostin (SOST), osteonectin (SPARC), Klotho (KL), receptor activator of nuclear factor κ-B ligand (RANKL), stanniocalcin-1 (STC1), stanniocalcin-2 (STC2), and Dickkopf-related protein 1 (DKK1).

32. The use of any one of claims 15 to 31, wherein calcification is induced in the first type and the second type of vascular smooth muscle cells using orthophosphate, a bone morphogenic protein, cholesterol, serum depletion, or microcurrent stimulation.

33. The use of any one of claims 15 to 32, wherein calcification is induced in the first type and the second type of vascular smooth muscle cells using orthophosphate.

34. The use of claim 33, wherein calcification is induced by adding orthophosphate to the culture media of the first type and the second type of vascular smooth muscle cells to a final concentration of about 2 mM to about 5 mM, preferably about 3 mM, in each culture medium.

35. The use of any one of claims 15 to 34, wherein the first type and the second type of vascular smooth muscle cells are cultured for a period of 3 days to 7 days starting from the induction of calcification.

36. The use of any one of claims 15 to 34, wherein the first type and the second type of vascular smooth muscle cells are cultured for a period of 7 days to 14 days starting from the induction of calcification.

37. The use of any one of claims 15 to 36, wherein the extent of calcification in the first type and in the second type of vascular smooth muscle cells is determined by detecting the formation of hydroxyapatite in the first type and the second type of vascular smooth muscle cells.

38. The use of any one of claims 15 to 37, wherein the extent of calcification in the first type and in the second type of vascular smooth muscle cells is determined using histochemical staining, immunohistochemical staining, or optical imaging with the contrast dye Cy-HABP-19.

39. The use of any one of claims 15 to 38, wherein the intracellular calcium concentration in the first type and in the second type of vascular smooth muscle cells is detected.

40. Use of a first type of cultured vascular smooth muscle cells and a second type of cultured vascular smooth muscle cells as a cell culture model of vascular calcification, wherein the first type and the second type of vascular smooth muscle cells originate from blood vessels having a different diameter.

41. The use of claim 40, wherein the first type and the second type of vascular smooth muscle cells are human cells.

42. The use of claim 40 or 41, wherein the first type and the second type of vascular smooth muscle cells are primary human vascular smooth muscle cells.

43. The use of any one of claims 40 to 42, wherein the first type of vascular smooth muscle cells originates from a blood vessel having a diameter that is at least about three times as large as the diameter of the blood vessel from which the second type of vascular smooth muscle cells originates.

44. The use of any one of claims 40 to 43, wherein the first type of vascular smooth muscle cells originates from a blood vessel having a diameter that is at least about 10 times as large as the diameter of the blood vessel from which the second type of vascular smooth muscle cells originates.

45. The use of any one of claims 40 to 44, wherein the first type of vascular smooth muscle cells originates from a blood vessel having a diameter that is at least about 50 times as large as the diameter of the blood vessel from which the second type of vascular smooth muscle cells originates.

46. The use of any one of claims 40 to 45, wherein the first type of vascular smooth muscle cells originates from a blood vessel having a diameter that is at least about 200 times as large as the diameter of the blood vessel from which the second type of vascular smooth muscle cells originates.

47. The use of any one of claims 40 to 46, wherein the first type of vascular smooth muscle cells originates from human aorta, and wherein the second type of vascular smooth muscle cells originates from human coronary artery.

48. The use of any one of claims 40 to 46, wherein the first type of vascular smooth muscle cells originates from a human artery, and wherein the second type of vascular smooth muscle cells originates from a human arteriole.

49. The use of any one of claims 40 to 46, wherein the first type of vascular smooth muscle cells originates from a human vein, and wherein the second type of vascular smooth muscle cells originates from a human venule.

50. The use of any one of claims 40 to 49, wherein calcification is induced in the first type and the second type of vascular smooth muscle cells.

51. The use of claim 50, wherein calcification is induced by the addition of orthophosphate, a bone morphogenic protein, cholesterol, serum depletion, or microcurrent stimulation.

52. The use of claim 50 or 51, wherein calcification is induced by the addition of orthophosphate.

53. The use of any one of claims 50 to 52, wherein calcification is induced by the addition of orthophosphate to the culture media of the first type and the second type of vascular smooth muscle cells to a final concentration of about 2 mM to about 5 mM, preferably about 3 mM, in each culture medium.

54. A method of analyzing vascular calcification, the method comprising:
culturing a first type of vascular smooth muscle cells and a second type of vascular smooth muscle cells, wherein the first type and the second type of vascular smooth muscle cells originate from blood vessels having a different diameter;
inducing calcification in the first type and in the second type of vascular smooth muscle cells; and
determining the extent of calcification in the first type and in the second type of vascular smooth muscle cells.

55. A method of identifying a calcification inhibitor, the method comprising:
culturing a first type of vascular smooth muscle cells and a second type of vascular smooth muscle cells, wherein the first type and the second type of vascular smooth muscle cells originate from blood vessels having a different diameter;
inducing calcification in the first type and in the second type of vascular smooth muscle cells;
adding a test agent to the first type and to the second type of vascular smooth muscle cells;
determining the extent of calcification in the first type and in the second type of vascular smooth muscle cells, both in the presence and in the absence of the test agent; and
identifying the test agent as a calcification inhibitor if the extent of calcification in the first type and/or in the second type of vascular smooth muscle cells is lower in the presence of the test agent than in the absence of the test agent.

56. A method of analyzing the effectiveness of a calcification inhibitor, the method comprising:
culturing a first type of vascular smooth muscle cells and a second type of vascular smooth muscle cells, wherein the first type and the second type of vascular smooth muscle cells originate from blood vessels having a different diameter;
inducing calcification in the first type and in the second type of vascular smooth muscle cells;
adding the calcification inhibitor to the first type and to the second type of vascular smooth muscle cells; and
determining the extent of calcification in the first type and in the second type of vascular smooth muscle cells.

57. The method of claim 56, wherein the calcification inhibitor is selected from a bisphosphonate, prednisolone, calcitriol, adenosine triphosphate, fibroblast growth factor 23, Klotho, foscarnet, microRNA 205, and a pharmaceutically acceptable salt or solvate of any of the aforementioned agents.

58. The method of claim 57, wherein the calcification inhibitor is a bisphosphonate which is preferably selected from etidronate, clodronate, tiludronate, pamidronate, neridronate, olpadronate, alendronate, ibandronate, risedronate, zoledronate, incadronate, minodronate, cimadronate, EB-1053, and a pharmaceutically acceptable salt or solvate thereof.

59. A method of analyzing the effect of electrical stimulation on vascular calcification, the method comprising:
culturing a first type of vascular smooth muscle cells and a second type of vascular smooth muscle cells, wherein the first type and the second type of vascular smooth muscle cells originate from blood vessels having a different diameter;
inducing calcification in the first type and in the second type of vascular smooth muscle cells;
applying an electrical current, preferably a microcurrent, to the first type and to the second type of vascular smooth muscle cells; and
determining the extent of calcification in the first type and in the second type of vascular smooth muscle cells.

60. The method of claim 59, the method comprising applying an electrical current of about 0.1 μA to about 100 μA, preferably about 0.5 μA to about 20 μA, at a frequency of about 1 mHz to about 25 Hz, to the first type and to the second type of vascular smooth muscle cells.

61. The method of any one of claims 54 to 60, wherein the first type and the second type of vascular smooth muscle cells are human cells.

62. The method of any one of claims 54 to 61, wherein the first type and the second type of vascular smooth muscle cells are primary human vascular smooth muscle cells.

63. The method of any one of claims 54 to 62, wherein the first type of vascular smooth muscle cells originates from a blood vessel having a diameter that is at least about three times as large as the diameter of the blood vessel from which the second type of vascular smooth muscle cells originates.

64. The method of any one of claims 54 to 63, wherein the first type of vascular smooth muscle cells originates from a blood vessel having a diameter that is at least about 10 times as large as the diameter of the blood vessel from which the second type of vascular smooth muscle cells originates.

65. The method of any one of claims 54 to 64, wherein the first type of vascular smooth muscle cells originates from a blood vessel having a diameter that is at least about 50 times as large as the diameter of the blood vessel from which the second type of vascular smooth muscle cells originates.

66. The method of any one of claims 54 to 65, wherein the first type of vascular smooth muscle cells originates from a blood vessel having a diameter that is at least about 200 times as large as the diameter of the blood vessel from which the second type of vascular smooth muscle cells originates.

67. The method of any one of claims 54 to 66, wherein the first type of vascular smooth muscle cells originates from human aorta, and wherein the second type of vascular smooth muscle cells originates from human coronary artery.

68. The method of any one of claims 54 to 66, wherein the first type of vascular smooth muscle cells originates from a human artery, and wherein the second type of vascular smooth muscle cells originates from a human arteriole.

69. The method of any one of claims 54 to 66, wherein the first type of vascular smooth muscle cells originates from a human vein, and wherein the second type of vascular smooth muscle cells originates from a human venule.

70. The method of any one of claims 54 to 69, wherein the method comprises inducing calcification in the first type and in the second type of vascular smooth muscle cells by adding orthophosphate, a bone morphogenic protein or cholesterol to the culture media of the first type and the second type of vascular smooth muscle cells, or by using serum depletion or microcurrent stimulation.

71. The method of any one of claims 54 to 70, wherein the method comprises inducing calcification in the first type and in the second type of vascular smooth muscle cells by adding orthophosphate to the culture media of the first type and the second type of vascular smooth muscle cells.

72. The method of claim 70 or 71, wherein orthophosphate is added to the culture media of the first type and the second type of vascular smooth muscle cells to a final concentration of about 2 mM to about 5 mM, preferably about 3 mM, in each culture medium.

73. The method of any one of claims 54 to 72, wherein the method comprises determining the extent of calcification in the first type and in the second type of vascular smooth muscle cells by detecting the formation of hydroxyapatite in the respective cells.

74. The method of any one of claims 54 to 73, wherein the method comprises determining the extent of calcification in the first type and in the second type of vascular smooth muscle cells by histochemical staining, immunohistochemical staining, or optical imaging with the contrast dye Cy-HABP-19.

75. The method of any one of claims 54 to 74, wherein the method further comprises determining the intracellular calcium concentration in the first type and in the second type of vascular smooth muscle cells.
